(19) United States
(12) Patent Application Publication
(10) Pub. No.: US 2020/0268807 A1
(43) Pub. Date: Aug. 27, 2020

(54) METHOD OF SYNTHESIZING ANTIMICROBIAL SILVER NANOPARTICLES USING PIGEON DUNG

(71) Applicant: KING SAUD UNIVERSITY, Riyadh (SA)

(72) Inventors: MANAL AHMED GASMELSEED AWAD, RIYADH (SA); MANAL MOHAMMED ALKHULAIFI, RIYADH (SA); ALI KANAKHIR ALDALBAHI, RIYADH (SA); NOURA SALEEM ALDOSARI, RIYADH (SA); SHAYKHA MOHAMMED ALZAHLY, RIYADH (SA)

(21) Appl. No.: 16/282,589

(22) Filed: Feb. 22, 2019

(51) Int. Cl. A61K 35/57; A61K 33/38; A61K 9/51

(52) U.S. Cl. CPC A61K 35/57; A61K 9/5192; A61K 9/51; A61K 33/38

(57) ABSTRACT

The method of synthesizing antimicrobial silver nanoparticles using pigeon dung includes collecting pigeon dung and suspending the pigeon dung in water to produce a pigeon dung aqueous extract, filtering the pigeon dung aqueous extract, adding a solution including a silver source to the pigeon dung aqueous extract to produce a mixture, and resting the mixture to allow silver nanoparticles to form. In an embodiment the antimicrobial pigeon dung nanoparticles may be incorporated in a pharmaceutical composition.

### BACKGROUND

### 1. Field

The disclosure of the present patent application relates to nanotechnology, and particularly to a method of synthesizing antimicrobial silver nanoparticles using pigeon dung.

### 2. Description of the Related Art

In materials science, nanomaterials have demonstrated unique size and morphology based characteristics. Nanotechnology is an emerging field demonstrating significant potential for the development of new medicines. The most common methods of producing nanoparticles are chemical or mechanical, including ball milling, thermal quenching, precipitation techniques, and vapor deposition. However, these methods are often costly, and may result in toxic byproducts.

Biological approaches for synthesizing nanoparticles can avoid many of the disadvantages associated with the chemical or mechanical synthesis methods.

Thus, a method of synthesizing antimicrobial silver nanoparticles using pigeon dung solving the aforementioned problems is desired.

### SUMMARY

The method of synthesizing antimicrobial silver nanoparticles using pigeon dung includes collecting pigeon dung and suspending the pigeon dung in water to produce a pigeon dung aqueous extract, filtering the pigeon dung aqueous extract, adding a solution including a silver source to the pigeon dung aqueous extract to produce a mixture, and resting the mixture to allow pigeon dung silver nanoparticles to form (generally indicated by a color change to dark brown).

An embodiment of the present subject matter is directed to a composition including pigeon dung nanoparticles and pigeon dung aqueous extract.

An embodiment of the present subject matter is directed to a pharmaceutical composition comprising the pigeon dung nanoparticles and a pharmaceutically acceptable carrier.

An embodiment of the present subject matter is directed to a method of making a pharmaceutical composition including mixing the pigeon dung nanoparticles with a pharmaceutically acceptable carrier.

These and other features of the present disclosure will become readily apparent upon further review of the following specification and drawings.

### BRIEF DESCRIPTION OF THE DRAWINGS

FIG. 1A depicts the zone of inhibition of the pigeon dung silver nanoparticles against Staphylococcus aureus.

FIG. 1B depicts the zone of inhibition of the pigeon dung silver nanoparticles against Staphylococcus aureus.

FIG. 1C depicts the zone of inhibition of the pigeon dung silver nanoparticles against Bacillus.

FIG. 1D depicts the zone of inhibition of the pigeon dung silver nanoparticles against Bacillus.

FIG. 1E: depicts the zone of inhibition of the pigeon dung silver nanoparticles against Escherichia coli.

FIG. 1F depicts the zone of inhibition of the pigeon dung silver nanoparticles against Escherichia coli.

FIG. 1G depicts the zone of inhibition of the pigeon dung silver nanoparticles against Pseudomonas aeruginosa.

FIG. 1H depicts the zone of inhibition of the pigeon dung silver nanoparticles against Pseudomonas aeruginosa.

FIG. 1I depicts the zone of inhibition of the pigeon dung silver nanoparticles against Alternaria alternata.

FIG. 1J depicts the zone of inhibition of the pigeon dung silver nanoparticles against Alternaria alternata.

FIG. 1K: depicts the zone of inhibition of the pigeon dung silver nanoparticles against Fusarium oxysporum.

FIG. 1L depicts the zone of inhibition of the pigeon dung silver nanoparticles against Fusarium oxysporum.

FIG. 1M depicts the zone of inhibition of the pigeon dung silver nanoparticles against Aspergillus flavus.

FIG. 1N depicts the zone of inhibition of the pigeon dung silver nanoparticles against Aspergillus flavus.

FIG. 1O depicts the zone of inhibition of the pigeon dung silver nanoparticles against Penicillium griseofulvum.

FIG. 1P depicts the zone of inhibition of the pigeon dung silver nanoparticles against Penicillium griseofulvum.

FIG. 2 depicts a UV-vis absorbance spectrum of pigeon dung silver nanoparticles.

FIG. 3 depicts a Zeta-sizer spectrum of pigeon dung silver nanoparticles.

FIG. 4A depicts a transmission electron micrograph of pigeon dung silver nanoparticles.

FIG. 4B depicts a transmission electron micrograph of pigeon dung silver nanoparticles.

FIG. 4C depicts a transmission electron micrograph of pigeon dung silver nanoparticles.

FIG. 4D depicts a transmission electron micrograph of pigeon dung silver nanoparticles.

FIG. 5 depicts an EDS analysis of pigeon dung silver nanoparticles.

Similar reference characters denote corresponding features consistently throughout the attached drawings.

### DETAILED DESCRIPTION OF THE PREFERRED EMBODIMENTS

A method of synthesizing antimicrobial silver nanoparticles using pigeon dung includes collecting pigeon dung and suspending the pigeon dung in water to produce a pigeon dung aqueous extract, filtering the pigeon dung aqueous extract, adding a solution including a silver source to the pigeon dung aqueous extract to produce a mixture, and resting the mixture to allow pigeon dung silver nanoparticles to form (generally indicated by a color change to dark brown).

As used herein, the term “about,” when used to modify a numerical value, means within ten percent of that numerical value.

In an embodiment, about 17 g of pigeon dung may be suspended in about 90 ml of distilled water to produce the pigeon dung aqueous extract.

In an embodiment, the silver source is AgNO₃. In an embodiment, about 0.012 g AgNO₃ is added to about 50 ml distilled water and stirred for a period of time to provide the solution including a silver source.

In an embodiment, the pigeon dung may be collected from pigeons found in Riyadh, Saudi Arabia.

In an embodiment, the filtering may include first filtering through gauze, then filtering the resulting filtrate through filter paper, such as Whatmann No. 1 filter paper.

An embodiment of the present subject matter is directed to a composition including pigeon dung nanoparticles and pigeon dung aqueous extract.

An embodiment of the present subject matter is directed to a pharmaceutical composition comprising the pigeon dung nanoparticles and a pharmaceutically acceptable carrier.

An embodiment of the present subject matter is directed to a method of making a pharmaceutical composition including mixing the pigeon dung nanoparticles with a pharmaceutically acceptable carrier. For example, the method of making a pharmaceutical composition can include mixing the pigeon dung nanoparticles under sterile conditions with a pharmaceutically acceptable carrier with preservatives, buffers, and/or propellants to create the pharmaceutical composition.

An embodiment of the present subject matter is directed to a pharmaceutical composition including the pigeon dung nanoparticles. To prepare the pharmaceutical composition, the pigeon dung nanoparticles, as the active ingredient, are intimately admixed with a pharmaceutically acceptable carrier according to conventional pharmaceutical compounding techniques. Carriers are inert pharmaceutical excipients, including, but not limited to, binders, suspending agents, lubricants, flavorings, sweeteners, preservatives, dyes, and coatings. In preparing compositions in oral dosage form, any of the pharmaceutical carriers known in the art may be employed. For example, for liquid oral preparations, suitable carriers and additives include water, glycols, oils, alcohols, flavoring agents, preservatives, coloring agents, and the like. Further, for solid oral preparations, suitable carriers and additives include starches, sugars, diluents, granulating agents, lubricants, binders, disintegrating agents, and the like.

The present compositions can be in unit dosage forms such as tablets, pills, capsules, powders, granules, ointments, sterile parenteral solutions or suspensions, metered aerosol or liquid sprays, drops, ampules, auto-injector devices or suppositories, for oral parenteral, intranasal, sublingual or rectal administration, or for administration by inhalation or insufflation. The active compound can be mixed under sterile conditions with a pharmaceutically acceptable carrier and, if required, any needed preservatives, buffers, or propellants. The composition can be presented in a form suitable for daily, weekly, or monthly administration. The pharmaceutical compositions herein will contain, per dosage unit, e.g., tablet, capsule, powder, injection, teaspoonful, suppository and the like, an amount of the active ingredient necessary to deliver an effective dose.

The following examples illustrate the present teachings.

### Example 1

### Synthesis of Pigeon Dung Nanoparticles

17 g of pigeon dung were collected from a pigeon shed in Riyadh, Saudi Arabia and suspended in 90 ml distilled water to produce a pigeon dung aqueous extract. The pigeon dung aqueous extract was then filtered through gauze and the filtrate was further filtered through Whatmann No. 1 filter paper, producing a filtered pigeon dung aqueous extract. The filtered pigeon dung aqueous extract was stored at 4° C. until use. A solution including a silver source was prepared by mixing about 0.012 g AgNO₃ with 50 ml distilled water and stirring for 15 minutes at 50° C. About 5 ml of the filtered pigeon dung aqueous extract was added to the silver source, producing a mixture. After about 30 minutes the mixture had changed color to dark brown, indicating the formation of silver nanoparticles.

### Example 2

### Characterization of Pigeon Dung Silver Nanoparticles

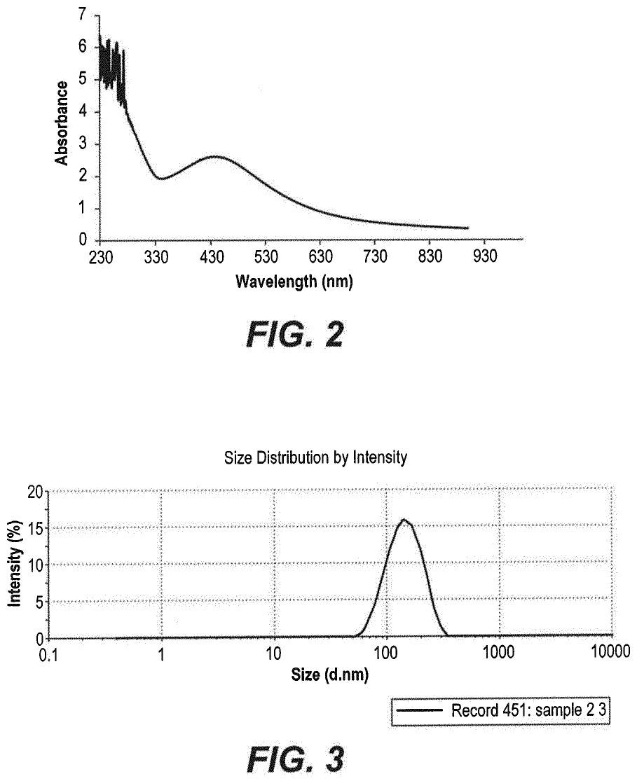

Pigeon dung silver nanoparticles were synthesized according to the method of Example 1 and the final mixture was observed for a further 30 minutes. Ultraviolet-Vis (UV-Vis) spectroscopy analysis was performed on a Perkin Elmer UV-Vis spectrometer. The formation of pigeon dung silver nanoparticles was also monitored via color change. After about an hour, the color of the mixture containing the pigeon dung silver nanoparticles changed to a reddish hue, attributed to the excitation of surface plasmon resonance (SPR) in silver nanoparticles. As shown in FIG. 2, a characteristic and well-defined SPR band peak appears in the UV-Vis spectrum at around λ454 nm.

Average sizes of the resulting pigeon dung silver nanoparticles were analyzed using a Zetasizer (ZEN36000, Molvern Instrument, UK). The average size of the pigeon dung silver nanoparticles was 135 nm, as illustrated in FIG. 3 and summarized in Table 1.

TABLE 1
Zetasizer Spectrum Data Summary

| | | | Size | % | St Dev |
|---|---|---|---|---|---|
| | | | (d · nm) | Intensity | (d · nm) |
| Z-Average (d · nm) | 135.0 | Peak 1 | 149.6 | 100.00 | 50.10 |
| Pdl | 0.398 | Peak 2 | 0.000 | 0.0 | 0.000 |
| Intercept | 0.927 | Peak 3 | 0.000 | 0.0 | 0.000 |
| Quality | Good | | | | |

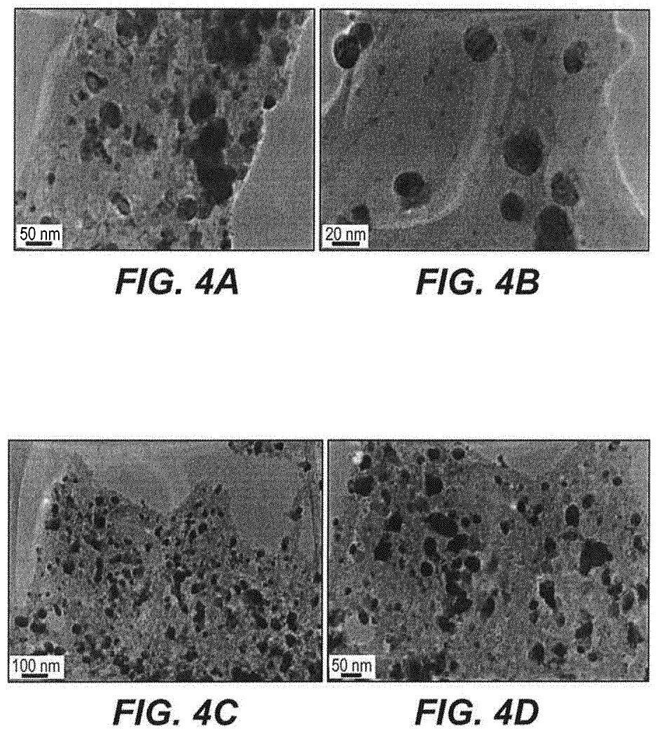

Transmission electron microscopy (TEM) (JEM-1400, JEOL, Japan) was employed to characterize the size, shape, and morphologies of the pigeon dung silver nanoparticles. The TEM images illustrated in FIG. 4A-4D depict the morphologies of the pigeon dung silver nanoparticles as distinctly variable and including many spherical and irregular shapes.

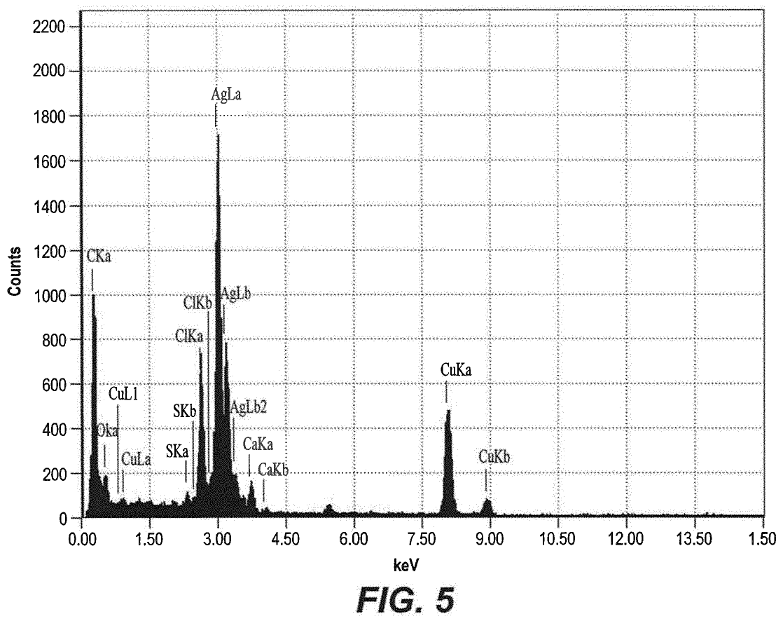

Elemental analysis of the pigeon dung silver nanoparticles was performed using an Energy-Dispersive X-ray (EDX). Energy dispersive spectroscopic analysis was performed to confirm the presence of silver in the pigeon dung nanoparticles. As illustrated in FIG. 5, this analysis confirmed the presence of silver in the pigeon dung nanoparticles.

### Example 3

### Evaluating the Antimicrobial Activity of the Pigeon Dung Nanoparticles

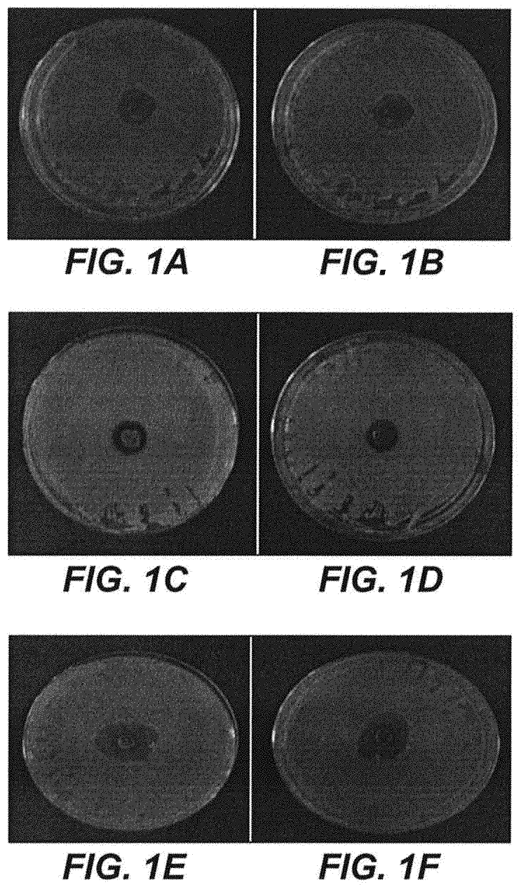

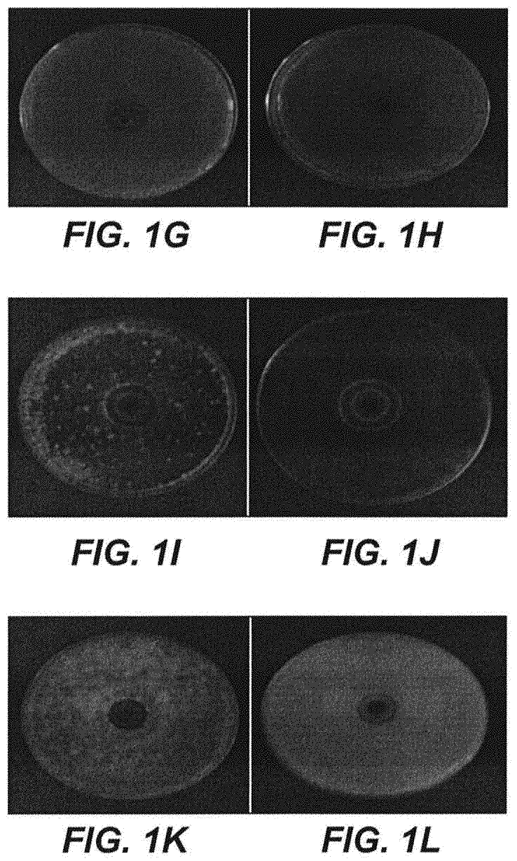

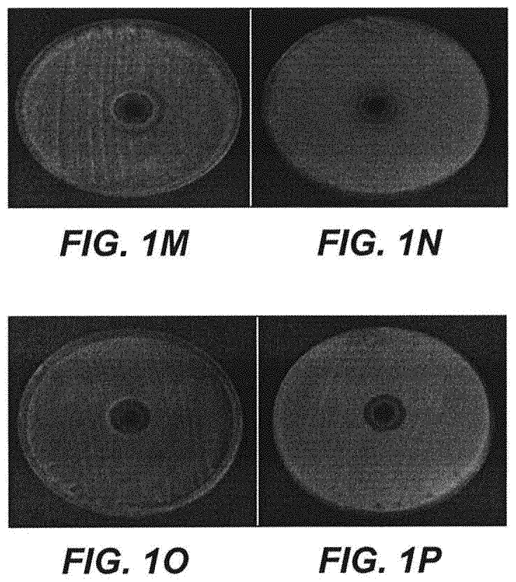

An agar well diffusion assay was performed to evaluate the antimicrobial activity of the pigeon dung silver nanoparticles against human pathogenic bacteria and fungi. The inoculum of the bacterial suspensions was swabbed on Muller Hinton agar plates, while the inoculum of the fungal suspensions was swabbed on Potato Dextrose Agar medium. Wells were cut into the middle of each plate, and 100 μl of the pigeon dung nanoparticle mixture prepared according to Example 1 was loaded into each well. The plates were incubated at 37° C. for 18-24 hours (bacteria) or 28° C. for 48-72 hours (fungi). After incubation, the plates were observed for formation of inhibition zones around the wells, indicating antimicrobial activity of the pigeon dung nanoparticles. The zone of inhibition was calculated by measuring the diameter of the inhibition zone formed around each well. These results are illustrated in FIG. 1A-1P and summarized in Table 2.

TABLE 2
Zone of Inhibition Measurements

| Organism | Zone of Inhibition | |
|---|---|---|
| Eschericia coli | 2.6 | cm |
| Staphylococcus aureus | 1.7 | cm |
| Bacillus | 1.5 | cm |
| Pseudomonas | 1.8 | cm |
| Aspergillus flavus | 2.7 | cm |
| Penicillium griseofulvum | 1.8 | cm |
| Fusarium oxysporium | 2 | cm |
| Alternaria alternata | 2.5 | cm |

It is to be understood that the method of synthesizing antimicrobial silver nanoparticles using pigeon dung is not limited to the specific embodiments described above, but encompasses any and all embodiments within the scope of the generic language of the following claims enabled by the embodiments described herein, or otherwise shown in the drawings or described above in terms sufficient to enable one of ordinary skill in the art to make and use the claimed subject matter.

1-2. (canceled)

3. A method of synthesizing pigeon dung nanoparticles, the method consisting of the sequential steps of:
(a) collecting pigeon dung;
(b) suspending 17 g of pigeon dung in 90 ml of water to form a pigeon dung aqueous extract;
(c) filtering the pigeon dung aqueous extract, wherein the filtering includes filtering the pigeon dung aqueous extract through gauze to provide a filtrate and subsequently filtering the filtrate through filter paper;
(d) preparing a silver nitrate aqueous solution, wherein 0.012 g AgNO₃ is mixed with 50 ml distilled water and stirred for 15 minutes at 50° C.;
(e) adding the solution to 5 ml of the filtered pigeon dung aqueous extract to form a mixture; and
(f) resting the mixture to allow pigeon dung silver nanoparticles to form, wherein the pigeon dung silver nanoparticles have an average diameter of 135 nm and an absorption band at 454 nm on the UV-Visible spectrum.

4-6. (canceled)

7. The method of synthesizing pigeon dung nanoparticles of claim 3, further comprising collecting the pigeon dung from a pigeon in Riyadh, Saudi Arabia.

8. (canceled)

9. Antimicrobial pigeon dung nanoparticles synthesized according to the method of claim 3.

10. (canceled)

11. A pharmaceutical composition comprising pigeon dung nanoparticles synthesized according to the method of claim 3 and a pharmaceutically acceptable carrier.

12. The pharmaceutical composition of claim 11, wherein the pharmaceutically acceptable carrier is selected from the group consisting of binders, suspending agents, lubricants, flavorings, sweeteners, preservatives, dyes, and coatings.

13. The pharmaceutical composition of claim 11, further comprising an additive selected from the group consisting of water, glycols, oils, alcohols, flavoring agents, preservatives, and coloring agents.

14. The pharmaceutical composition of claim 11, further comprising an additive selected from the group consisting of starches, sugars, diluents, granulating agents, lubricants, binders, and disintegrating agents.

15. The pharmaceutical composition of claim 11, wherein the pharmaceutical composition is compounded in a unit dosage form, the unit dosage form selected from the group consisting of tablets, pills, capsules, powders, granules, ointments, sterile parenteral solutions, sterile suspensions, metered aerosol sprays, metered liquid sprays, drops, ampules, auto-injector devices, and suppositories.
